(19) United States
(12) Patent Application Publication
(10) Pub. No.: US 2013/0156750 A1
(43) Pub. Date: Jun. 20, 2013

(54) Compositions and Methods for Using Human YKL-40 to Treat Acute Lung Injury

(71) Applicant: Jack A. Elias, Woodbridge, CT (US)

(72) Inventor: Jack A. Elias, Woodbridge, CT (US)

(73) Assignee: YALE UNIVERSITY, New Haven, CT (US)

(21) Appl. No.: 13/704,400

(22) PCT Filed: Jun. 16, 2011
(86) PCT No.: PCT/US11/40638

§ 371 Date: Mar. 7, 2013

Related U.S. Application Data
(60) Provisional application No. 61/397,766, filed on Jun. 16, 2010.

(51) Int. Cl. A61K 38/47

(52) U.S. Cl. CPC A61K 38/47
USPC 424/94.61; 435/18; 435/7.92

(57) ABSTRACT

The invention includes methods of treating oxidant-mediated acute lung injury in a subject by administration of a chitinase-like protein molecule, or an activator thereof. The invention also includes methods of assessing the level of a chitinase-like protein molecule in a subject as a marker of the prognosis of a subject suffering from acute lung injury.

### BACKGROUND OF THE INVENTION

Supplemental oxygen is commonly administered to patients with significant pulmonary and cardiac disease to increase the delivery of oxygen to peripheral tissues. However, very high concentrations of oxygen (fractional inspired concentrations 50%) for prolonged periods cause hyperoxic acute lung injury (HALO). This response is characterized by endothelial and epithelial injury and enhanced alveolar capillary protein leak (Klekamp et al., 1999, Am J Pathol 154:823-831; Warner et al., 1998, Am J Physlol 275:L110-L117; Horowitz and Davis, 1997, McDonald J M, editor. Lung growth and development. New York: Marcel Dekker, Inc., pp. 577-610; Slutsky, 1999, Chest 116:9S-15S; Conte J et al., 2000, J Clin Invest 106:783.791; Waxman et al., 1998, J Clin Invest 101:1970-1982), Studies of this response have led to the free radical theory that suggests that, in 100% O₂, lung cells poison themselves by producing an excess of reactive oxygen species (He et al., 2005, J Clin Invest 115:1039-1048; Barazzone et al., 2000, Am J Respir Cell Mol Biol 22:517-519; Freeman et al., 1982, Lab Invest 47; 412-426). Recent studies have added to this pathogenic paradigm by demonstrating that reactive oxygen species mediate their effects, in part, by inducing an endothelial and epithelial cell death response with features of apoptosis and necrosis (Waxman et al., 1998, J Clin Invest 101:1970-1982; He et al., 2005, J Clin Invest 115:1039-1048; Ward et al., 2000, Am J Respir Cell Mol Biol 22:535-542; Mantell et al., 1999, Ann N Y Acad Sci 887:171-180; Barazzone et al., 1998, Am J Respir Cell Mol Biol 19:573-581; O'Reilly et al., 2000, Lab Invest 80:1845-1854; Wang et al., 2003, J Biol Chem 278:29184-29191). In spite of the obvious importance of pathways that regulate these toxic responses, and the well-documented variations in the ability of inbred mice to withstand exposure to 100% O₂ (Waxman et al., 1998, J Clin Invest 101:1970.1982; He et al., 2005, J Clin Invest 115:1039-1048; Ward et al., 2000, Am J Respir Cell Mol Biol 22:535-542; Paine et al., 2003, Am J Pathol 163:2397-2406), the endogenous mechanisms that contribute to the control of these responses have not been adequately defined.

The evolutionarily conserved 18-glycosyl-hydrolase family contains true chitinases and molecules that lack chitinase activity (Boot et al., 2001, J Biol Chem 276:6770-6778; Chupp et al., 2007, N Engl J Med 357:2016-2027; Kawada et al., 2007, Keio J Med 56:21-27; Zhu et al., 2004, Science 304:1678-1682). Much of the research in this area has focused on chitinases such as acidic mammalian chitinase, which plays a critical role in the life cycle of parasites and the pathogenesis of T helper cell (Th) 2 and antiparasite responses (Kawada et al., 2007, Keio J Med 56:21-27; Zhu et al., 2004, Science 304:1678-1682; Reese et al., 2007, Nature 447:92-96). However, the majority of the 18-glycosyl-hydrolase family members are chitinase-like protein molecules, which, as a result of mutations in their highly conserved enzyme sites, do not contain chitinase activity. Breast regression protein (BRP)-39 and its human homolog, YKL-40 (also called chitinase-3-like 1 and human cartilage glycoprotein-39) (Hakala et al., 1993, J Biol Chem 268:25803-25810; Rejman et al., 1988, Biochem Biophys Res Commun 150:329-334; Shackleton et al., 1995, J Biol Chem 270:13076-13083) are the prototypes of these enzymatically deficient chitinase-like protein molecules. They are produced by a variety of cells, including neutrophils, monocytes, and macrophages (Kawada et al., 2007, Keio J Med 56:21-27; Hakala et al., 1993, J Biol Chem 268:25803-25810; Johansen et al., 2006, Dan Med Bull 53:172-209). In addition, increased levels of YKL-40 protein and/or mRNA have been noted in patients with a wide spectrum of pathologies, including bacterial infections, rheumatoid arthritis, osteoarthritis, giant cell arteritis, sarcoidosis, scleroderma, diabetes, atherosclerosis, inflammatory bowel disease, and a variety of malignancies (Kawada et al., 2007, Keio J Med 56:21-27; Hakala et al., 1993, J Biol Chem 268:25803-25810, Johansen et al., 2006, Dan Med Bull 53; 172-209; Johansen et al., 2000, J Hepatol 32:911-920; Ostergaard et al., 2002, Clin Diagn Lab Immunol 9:598-604; Knudsen et al., 2006, Scand J Rheumatol 35:489-491; Kucur et al., 2007, Coron Artery Dis 18:391-396; Rathcke et al., 2006, Inflamm Res 55:221-227). In many of these disorders, the levels of YKL-40 reflect the activity and natural history of the disease (Chupp et al., 2007, N Engl J Med 357:2016-2027; Knudsen et al., 2006, Scand J Rheumatol 35:489-491; Kucur et al., 2007, Coron Artery Dis 18:391-396; Rathcke et al., 2006, Inflamm Res 55:221-227). This is illustrated by studies which have demonstrated that elevated levels of YKL-40 are seen in patients with asthma, which correlate with the levels of lung tissue YKL-40 and disease severity (Chupp et al., 2007, N Engl J Med 357:2016-2027). These studies also highlighted polymorphisms in the chitinase-3-like 1 gene that correlated with the levels of circulating YKL-40, the presence of asthma, and compromised lung function (Ober et al., 2008, N Engl J Med 358:1682-1691). Surprisingly, although oxidant-induced injuries are believed to contribute to the pathogenesis of many of these responses, the relationship(s) between BRP-39/YKL-40 and oxidant injury has not been investigated.

Acute lung injury (ALI) and its most severe form, acute RDS (ARDS), are devastating clinical syndromes affecting greater than 200,000 patients per year in the United States alone (Rubenfeld et al., 2005, N Engl J Med 353:1685-1693). Despite recent advances in therapy, the mortality for ARDS remains in the 25-50% range (Gao et al., 2009, Am J Physiol Lung Cell Mol Physiol 296:L713-L725). On the other end of the spectrum, bronchopulmonary dysplasia (BPD), in which HALL is a critical contributing factor, is the commonest chronic lung disease in infants (Bhandari et al., 2009, Pediatrics 123:1562-1573). There are currently no specific or effective interventions that prevent or ameliorate established BPD, and no established biomarkers that predict its occurrence in premature infants (Bhandari et al., 2009, Pediatrics 123:1562-1573).

There is thus a need in the art for compositions and methods of treating acute lung injury. The present invention addresses this unmet need in the art.

### BRIEF SUMMARY OF THE INVENTION

In one embodiment, the invention relates to a method of treating acute lung injury in a subject that involves administering an effective amount of a chitinase-like protein molecule, or an activator of a chitinase-like protein molecule, to the subject to treat the subject. In some embodiments, the chitinase-like protein molecule is YKL-40. In certain embodiments, the acute lung injury that is treated is an oxidant-mediated acute lung injury. In some embodiments, the subject is a human.

In another embodiment, the invention relates to a method of preventing acute lung injury in a subject that involves administering an effective amount of a chitinase-like protein molecule, or an activator of a chitinase-like protein molecule, to the subject, to prevent acute lung injury in the subject. In some embodiments, the chitinase-like protein molecule is YKL-40. In certain embodiments, the acute lung injury that is prevented is an oxidant-mediated acute lung injury. In some embodiments, the subject is a human.

In a further embodiment, the invention relates to a method of determining the severity of acute lung injury in a subject, involving the steps of 1) obtaining a sample from the subject, where the subject has, or is suspected of having, acute lung injury, 2) determining in the sample the level of at least one chitinase-like protein molecule, 3) comparing the level of the at least one chitinase-like protein molecule in the sample with the level in a control or reference standard, where the difference in the level of the at least one chitinase-like protein molecule between the sample and the control or reference standard is a measure of the severity of acute lung injury in the subject. In some embodiments, the chitinase-like protein molecule is YKL-40. In certain embodiments, the acute lung injury is an oxidant-mediated acute lung injury. In some embodiments, the subject is a human.

In another embodiment, the invention relates to a method of evaluating the progression of acute lung injury in a subject, involving the steps of: 1) obtaining a sample from the subject, where the subject has, or is suspected of having, acute lung injury, 2) determining in the sample the level of at least one chitinase-like protein molecule, 3) comparing the level of the at least one chitinase-like protein molecule in the sample with the level in a control or reference standard at a first time point, 4) comparing the level of the at least one chitinase-like protein molecule in the sample with the level in a control or reference standard at a second time point, where the difference in the level of the at least one chitinase-like protein molecule between the sample and the control or reference standard at the first time point and the second time point is a measure of the progression of acute lung injury in the subject. In some embodiments, the chitinase-like protein molecule is YKL-40. In certain embodiments, the acute lung injury is an oxidant-mediated acute lung injury. In some embodiments, the subject is a human.

In still a further embodiment, the invention relates to a method of evaluating the effect of a treatment of acute lung injury in a subject, involving the steps of: 1) obtaining a sample from the subject, where the subject has, or is suspected of having, acute lung injury, determining in the sample the level of at least one chitinase-like protein molecule, comparing the level of the at least one chitinase-like protein molecule in the sample with the level in a control or reference standard before treatment, comparing the level of the at least one chitinase-like protein molecule in the sample with the level in a control or reference standard after treatment, where the difference in the level of the at least one chitinase-like protein molecule between the sample and the control or reference standard before treatment and after treatment is a measure of the effect of the treatment of acute lung injury on the subject. In some embodiments, the chitinase-like protein molecule is YKL-40. In certain embodiments, the acute lung injury is an oxidant-mediated acute lung injury. In some embodiments, the subject is a human.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIGS. 1A through 1G, is a series of images depicting the results of experiments evaluation hyperoxia regulation of breast regression protein (BRP)-39. Mice were exposed to room air (RA) or 100% O₂ for up to 72 hours (FIG. 1A through 1E). FIG. 1A depicts the levels of BRP-39 mRNA evaluated by real-time reverse transcriptase-polymerase chain reaction (RT-PCR), FIG. 1B depicts BRP-39 protein accumulation assessed via Western analysis. FIGS. 1C and 1E depict BRP-39 protein accumulation assessed via ELISA of bronchoalveolar lavage fluid and lung lysates, respectively. FIG. 1D depicts the results of an experiment employing immunohistochemistry to localize the BRP-39. The closed arrows refer to alveolar type II cells; the open arrow refers to macrophage. BEAS-2B cells were exposed to 95% O₂ in the presence or absence of N-acetyl-L-cysteine (NAC). FIG. 1F depicts the results of an experiment using real-time RT-PCR to evaluate the effects of O₂ exposure on YKL-40 expression. FIG. 1G depicts the effects of NAC in this setting. The values in FIGS. 1A, 1C, and 1E represent the mean (±SEM) of evaluations in a minimum of five animals. The values in FIGS. 1F and 1G represent the mean (±SEM) of triplicate experiments. The value in FIG. 1B is representative of two separate experiments. The value in FIG. 1D is representative of four similar evaluations. Scale bar=50 μm. *P<0.05, **P<0.01.

FIG. 2, comprising FIGS. 2A through 2D, is a series of images depicting the results of experiments evaluating the role of breast regression protein (BRP)-39 in hyperoxia-induced vascular permeability and premature death. FIG. 2A depicts the results of experiments quantifying bronchoalveolar lavage (BAL) protein in wild-type (WT) (+/+) mice exposed to 100% O₂ up to 72 hours. FIG. 2B depicts the results of experiments assessing BAL protein in WT and BRP-39^{-/-} mice exposed to 100% O₂ for 72 hours. FIG. 2C depicts experiments assessing survival of C57BL/6 mice exposed to 100% O₂. FIG. 2D depicts experiments assessing survival of Balb/c mice exposed to 100% O₂. The values in FIGS. 2A and 2B are the mean (±SEM) of evaluations of a minimum of five animals, and are representative of two separate experiments. Data in FIGS. 2C and 2D represent assessments of a minimum of eight mice. NS, not significant. *P<0.05, **P<0.01.

FIG. 3, comprising FIGS. 3A through 3E, is a series of images depicting the results of experiments examining the role of breast regression protein (BRP)-39 in hyperoxia-induced inflammation and chemokine production. FIG. 3A depicts the results of experiments measuring bronchoalveolar lavage (BAL) total cell recovery after wild-type (WT) (+/+) and BRP-39^{-/-} mice were exposed to 100% O₂ for 72 hours. FIG. 3B depicts the results of experiments assessing differential cell recovery. FIG. 3C depicts lung histology of hematoxylin and eosin stain. FIG. 3D depicts the results of experiments assessing the levels of BAL KC/CXCLI and FIG. 3E depicts the results of experiments assessing the level of MCP-1/CCL-2. The values in FIGS. 3A, 3B, 3D and 3E represent the mean (±SEM) evaluations of a minimum of five animals, and are representative of two separate experiments. The value in FIG. 3C is representative of five similar evaluations. NS, nonsignificant. Scale bar, 100 μm. *P<0.05.

FIG. 4, comprising FIGS. 4A through 4D, is a series of images depicting the results of experiments evaluating the role of breast regression protein (BRP)-39 in hyperoxia-induced oxidant and DNA injury. FIG. 4A depicts the results of experiments evaluating wild-type (WT) (+/+) and BRP-39−/− mice exposed to room air (RA) or 100% O₂ for 72 hours, and subjected to 8-hydroxy-2′deoxyguanosine (8-OHdG). The closed arrows refer to airway and alveolar type II epithelial cells; the open arrows refer to macrophage. FIG. 4B depicts the results of experiments using terminal deoxynucleotidyl transferase-mediated dUTP nick end labeling (TUNEL). The closed arrows refer to alveolar type H cells; the open arrows refer to macrophages. FIG. 4C depicts the results of experiments counting TUNEL-positive cells, FIG. 4D depicts the results of experiments using double-label immunohistochemistry with cell-specific antibodies showing that TUNEL-positive apoptotic cells were localized. CC10 shows airway epithelial cells; pro-SPC shows alveolar type II cells. FIG. 4D depicts the results of experiments using TUNEL staining. Arrows refer to double-stained cells. FIGS. 4A and 4B are representative composites of five similar evaluations. The values in FIG. 4C are the mean (6SEM) of evaluations of a minimum of five animals, and are representative two separate experiments. The value in FIG. 4D is representative of two separate experiments. Scale bar, 50 μm. NS, nonsignificant. *P<0.05.

FIG. 5, comprising FIGS. 5A through 5I, is a series of images depicting the effects of transgenic YKL-40 on hyperoxia-induced bronchoalveolar lavage (BAL) and tissue responses. Wild-type (WT) (+/+) mice, mice with null mutations of breast regression protein (BRP)-39 (BRP-39−/−) mice, and BRP-39−/− mice that express transgenic YKL-40 only in respiratory epithelium (BRP-39−/−/YKL-40+ mice) were exposed to room air (RA) or 100% O₂. FIGS. 5A and 5I depict the results of experiments assessing survival. FIG. 5B depicts the results of experiments measuring BAL total cells after 72 hours of hyperoxia. FIG. 5C depicts differential cell recovery. FIG. 5D depicts the levels of BAL KC/CXCL1, FIG. 5E depicts the results of experiments measuring the level of MCP-1/CCL-2. FIG. 5F depicts lung tissue histology of hematoxylin and eosin stain. FIG. 5G depicts the results of experiments measuring the percentage of terminal deoxynucleotidyl transferase-mediated dUTP nick end labeling (TUNEL)-positive cells. FIG. 5H depicts the results of experiments evaluating Capase-3 activation and ICAD cleavage. The data in FIGS. 5A and 5I represent assessments in a minimum of eight mice. The values in FIGS. 5B through 5E and 5G are the mean (±SEM) of evaluations in a minimum of five animals, and are representative of two separate experiments. FIGS. 5F and 5H are representative of four similar evaluations. Scale bars, 100 μm. *P<0.05.

FIG. 6, comprising FIGS. 6A through 6D, is a series of images depicting the role of caspase-3 in breast regression protein (BRP)-39 regulation of hyperoxia-induced responses. Wild-type (WT) (+/+) mice, BRP-39^{-/-} mice, caspase-3^{-/-} mice, and BRP-39^{-/-}/caspase-3^{-/-} mice were exposed to room air (RA) or 100% O₂ for 72 hours. FIG. 6A depicts the results of experiments measuring bronchoalveolar lavage (BAL) total cell recovery. FIG. 6B depicts the results of experiments measuring BAL macrophage recovery. FIG. 6C depicts the results of experiments counting BAL neutrophil recovery. FIG. 6D depicts the results of experiments assessing BAL protein. The values represent the mean (±SEM) of evaluations in a minimum of five animals, and are representative of two separate experiments. Casp 3=caspase−3. *P<0.05.

FIG. 7 depicts the results of experiments measuring YKL-40 protein levels in tracheal aspirates from premature babies with respiratory failure requiring mechanical ventilation and O₂ supplementation. The levels of YKL-40 were evaluated by ELISA. The values represent the mean (±SEM) of evaluations in patients that developed bronchopulmonary dysplasia (BPD) or died (n=5) and those that did not develop these complications (No BPD; n=4). *P<0.05.

### DETAILED DESCRIPTION

The invention includes methods of treating or preventing oxidant-mediated acute lung injury in a subject by administration of a chitinase-like protein molecule. As the data disclosed elsewhere herein demonstrate, an increased level of a chitinase-like protein molecule in a subject is protective of oxidant-mediated lung injury. One example of oxidant-mediated acute lung injury treatable or preventable by the compositions and methods of the invention is hyperoxic acute lung injury. The compositions and methods described herein are useful in treating oxidant-mediated lung injury in subjects who are, or may be, exposed to oxygen levels greater than about 21%, such as, for example, subjects having asthma, chronic obstructive pulmonary disease, interstitial lung disease, chronic obstructive lung disease, chronic bronchitis, eosinophilic bronchitis, eosinophilic pneumonia, pneumonia, inflammatory bowel disease, atopic dermatitis, atopy, allergy, allergic rhinitis, idiopathic pulmonary fibrosis, scleroderma, emphysema, bronchopulmonary dysplasia, acute respiratory distress syndrome and the like. In certain embodiments, the subjects treated with the inventive compositions and methods are exposed to oxygen levels greater than about 50%. In some embodiments, the acute lung injury is, including hyperoxic acute lung injury (HALI).

The data disclosed herein demonstrate that increased expression, presence and/or activity of a chitinase-like protein molecule is associated with and/or mediates various etiologies. Further, the data disclosed herein demonstrate, surprisingly, that increasing the expression, presence and/or activity of a chitinase-like protein molecule, such as, but not limited to, YKL-40, provides a protective effect and therapeutic benefit and prevents, diminishes or treats oxidant-mediated acute lung injury. In some embodiments of the invention, the oxidant-mediated acute lung injury is hyperoxic acute lung injury. Indeed, the data demonstrate that administration of a chitinase-like protein molecule before or during the onset of the disease state serves to prevent or diminish the severity of acute lung injury. Accordingly, the present invention provides a novel method whereby administration of a chitinase-like protein molecule, or an activator thereof, in a subject who is, or may be, exposed to oxygen levels greater than about 21%, treats and/or prevents acute lung injury.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

By the term “applicator” as the term is used herein, is meant any device including, but not limited to, a hypodermic syringe, a pipette, an intravenous infusion, topical cream and the like, for administering the chitinase-like protein molecule or a compound that increases the expression, level or activity of a chitinase-like protein molecule to a subject.

“Chitinase,” as used herein, refers to a family of polypeptides comprising microbial and mammalian chitinases. A chitinase of the present invention demonstrates detectable chitinase activity, in that it specifically cleaves chitin in an endochitinase manner.

“Chitinase-like protein molecule,” as the term is used herein, encompasses a family of polypeptides comprising proteins that are defined by a certain degree of homology to known chitinases, but may not demonstrate detectable chitinase activity. Chitinase-like protein molecules include, but are not limited to acidic mammalian chitinase (also referred to as eosinophil chemotactic cytokine and exemplified by GenBank Ace. No. AF290003 and No, AF29004), YM1 (also known as chitinase 3-like 3, ECF-L precursor, as exemplified by GenBank Ace. No. M94584), YM2 as exemplified by GenBank Ace. No. AF461142, oviductal glycoprotein 1 as exemplified by GenBank Ace, No. XM_{—}131100, cartilage glycoprotein 1 (also referred to as BRP-39, chitinase 3-like 1, GP-1-39, YKL-40 as exemplified by GenBank Ace. No. X93035), chitotriosidase as exemplified by GenBank Acc. No. NM_{—}003465, oviductal glycoprotein 1 (also referred to as mucin 9, oviductin and as exemplified by GenBank Ace. No. NM_{—}002557), cartilage glycoprotein-39 (also known as chitinase 3-like 1, GP-39, YKL-40, as exemplified by GenBank Ace. No. NM_{—}001276), and chondrocyte protein 39 (also known as chitinase 3-like 2, YKL-39, as exemplified by GenBank Ace. No. NK_{—}04000). Thus, the skilled artisan would appreciate, once armed with the teachings provided herein, that the present invention encompasses chitinase-like protein molecules that possess detectable chitinase activity as well as those similar to the afore-mentioned molecules in that the potential chitinase-like protein molecules shares substantial sequence homology to the family of proteins. The invention is not limited to these particular chitinase-like protein molecules; rather, the invention includes other chitinase-like protein molecules that share substantial homology with them and/or which possess detectable chitinase activity, and encompasses such molecules known in the art as well as those discovered in the future.

By “chitinase-like protein molecule activator” is meant a compound that detectably increase the level of a chitinase-like protein molecule in a cell or tissue when compared to the level of the chitinase-like protein molecule in an otherwise identical cell or tissue in the absence of the compound. The level of the chitinase-like protein molecule includes, but is not limited to, the level of expression of a nucleic acid encoding the molecule, the level of chitinase-like protein molecule detectable, and/or the level of chitinase-like protein molecule activity.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “fragment,” as applied to a nucleic acid or polypeptide, refers to a subsequence of a larger nucleic acid or polypeptide. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between). A “fragment” of a polypeptide can be at least about 15 nucleotides in length; for example, at least about 50 amino acids to about 100 amino acids; at least about 100 to about 500 amino acids, at least about 500 to about 1000 amino acids, at least about 1000 amino acids to about 1500 amino acids; or about 1500 amino acids to about 2500 amino acids; or about 2500 amino acids (and any integer value in between).

As used herein, the terms “gene” and “recombinant gene” refer to nucleic acid molecules comprising an open reading frame encoding a polypeptide. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

“Homologous” as used herein, refers to the subunit sequence similarity between two polymeric molecules, e.g. between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g. if half (e.g., five positions in a polymer ten subunits in length) of the positions in two compound sequences are homologous then the two sequences are 50% homologous, if 90% of the positions, e.g. 9 of 10, are matched or homologous, the two sequences share 90% homology. By way of example, the DNA sequences 5′-ATTGCC-3′ and 5′-TATGGC-3′ share 75% homology.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a subject. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized, upon the other. By way of example, a promoter operably linked to the coding region of a gene is able to promote transcription of the coding region.

Preferably, when the nucleic acid encoding the desired protein further comprises a promoter/regulatory sequence, the promoter/regulatory sequence is positioned at the 5′ end of the desired protein coding sequence such that it drives expression of the desired protein in a cell. Together, the nucleic acid encoding the desired protein and its promoter/regulatory sequence comprise a “transgene.”

“Constitutive” expression is a state in which a gene product is produced in a living cell under most or all physiological conditions of the cell.

“Inducible” expression is a state in which a gene product is produced in a living cell in response to the presence of a signal in the cell.

A “recombinant polypeptide” is one, which is produced upon expression of a recombinant polynucleotide.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

As used herein, the term “transgenic subject” means a subject, the germ cells of which, comprise an exogenous nucleic acid.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a subject. In certain non-limiting embodiments, the subject is a human.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient. Disease and disorder are used interchangeably herein.

A “prophylactic” treatment is a treatment administered to a subject who does not exhibit signs of a disease or exhibits only early signs of the disease for the purpose of decreasing the risk of developing pathology associated with the disease.

“Preventing” a disease, as the term is used herein, means that the onset of the disease is delayed, and/or that the symptoms of the disease will be decreased in intensity and/or frequency, when an increased level of chitinase-like protein molecule is administered compared with the onset and/or symptoms in the absence of the increased level of chitinase-like protein molecule.

The phrase “therapeutically effective amount,” as used herein, refers to an amount that is sufficient or effective to prevent or treat (delay or prevent the onset of, prevent the progression of, inhibit, decrease or reverse) oxidant-mediated acute lung injury.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention partly relates to the discovery that hyperoxia is a potent inhibitor of BRP-39 expression and production, and that BRP-39 and YKL-40 inhibit the toxic effects of oxidant-mediated acute lung injury. Accordingly, the invention provides compositions and methods for treating oxidant-mediated acute lung injury.

### Methods of Treating and Preventing Acute Lung Injury

The present invention includes methods of treating oxidant-mediated acute lung injury by increasing the level of chitinase-like protein in the lung tissues of a subject, preferably a human. This is because, as would be appreciated by one skilled in the art when provided with the disclosure herein, increasing the expression and/or activity of a chitinase-like protein molecule serves as a treatment for acute lung injury, including acute lung injury mediated by an oxidant. That is, the data disclosed herein demonstrate that administration of a chitinase-like protein molecule, or an activator thereof, in a model of acute lung injury associated with, or mediated by, an oxidant, treats the disease after it has become established. Further, the present invention relates to the discovery that chitinase-like protein molecules and chitinase-like protein molecule mRNA are present in increased levels in subjects that cope better when exposed to an oxidant, such as when breathing oxygen at a level greater than about 21%. Thus, the present invention relates to treating of such diseases using a chitinase-like protein molecule, or an activator thereof, including, but not limited to YKL-40.

It would be understood by one skilled in the art, based upon the disclosure provided herein, that an increase in the level of a chitinase-like protein molecule encompasses the increase of chitinase-like protein molecule expression. Additionally, the skilled artisan would appreciate, once armed with the teachings of the present invention, that increase in the level of a chitinase-like protein molecule includes an increase in the chitinase-like protein molecule activity in a cell. Thus, increasing the level or activity of a chitinase-like protein molecule includes, but is not limited to, increasing transcription, translation, or both, of a nucleic acid encoding a chitinase-like protein molecule; and it also includes increasing any activity of the chitinase-like protein molecule as well.

Activation of a chitinase-like protein molecule can be assessed using a wide variety of methods, including those disclosed herein, as well as methods well-known in the art or to be developed in the future. That is, the routineer would appreciate, based upon the disclosure provided herein, that increasing the level or activity of a chitinase-like protein molecule can be readily assessed using methods that assess the level of a nucleic acid encoding a chitinase-like protein molecule (e.g., mRNA) and/or the level of a chitinase-like protein molecule present in a cell or fluid.

One skilled in the art, based upon the disclosure provided herein, would understand that the invention is useful in treating oxidant-mediated king injury in subjects who, for example, are being or will be, exposed to oxygen levels greater than 21%, such as subjects having asthma, chronic obstructive pulmonary disease, interstitial lung disease, chronic obstructive lung disease, chronic bronchitis, eosinophilic bronchitis, eosinophilia pneumonia, pneumonia, inflammatory bowel disease, atopic dermatitis, atopy, allergy, allergic rhinitis, idiopathic pulmonary fibrosis, scleroderma, emphysema, bronchopulmonary dysplasia, acute respiratory distress syndrome and the like. Further, the skilled artisan would further appreciate, based upon the teachings provided herein, that the oxidant-mediated acute lung injuries treatable by the compositions and methods described herein encompass any oxidant-induced acute lung injury, including oxidants other than breathing air at a level higher than 21%.

A chitinase-like protein molecule activator can include, but should not be construed as being limited to, a chemical compound, a protein, a peptidomemetic, an antibody, a ribozyme, and an antisense nucleic acid molecule. One of skill in the art would readily appreciate, based on the disclosure provided herein, that a chitinase-like protein molecule activator encompasses a chemical compound that increase the level or activity of a chitinase-like protein molecule. Additionally, a chitinase-like protein molecule activator encompasses a chemically modified compound, and derivatives, as is well known to one of skill in the chemical arts.

Further, one of skill in the art would, when equipped with this disclosure and the methods exemplified herein, appreciate that a chitinase-like protein molecule activator includes such activators as discovered in the future, as can be identified by well-known criteria in the art of pharmacology, such as the physiological results of activation of a chitinase-like protein molecule as described in detail herein and/or as known in the art. Therefore, the present invention is not limited in any way to any particular chitinase-like protein molecule activator as exemplified or disclosed herein; rather, the invention encompasses those activators that would be understood by the mutineer to be useful as are known in the art and as are discovered in the future.

Further methods of identifying and producing a chitinase-like protein molecule activators are well known to those of ordinary skill in the art, including, but not limited, obtaining an activator from a naturally occurring source (i.e., Streptomyces sp., Pseudomonas sp., Stylotella aurantium). Alternatively, a chitinase-like protein molecule activator can be synthesized chemically. Further, the routineer would appreciate, based upon the teachings provided herein, that a chitinase-like protein molecule activator can be obtained from a recombinant organism. Compositions and methods for chemically synthesizing chitinase-like protein molecule activators and for obtaining them from natural sources are well known in the art and are described in the art.

One of skill in the art will appreciate that an activator can be administered as a small molecule chemical, a protein, a nucleic acid construct encoding a protein, or combinations thereof. Numerous vectors and other compositions and methods are well known for administering a protein or a nucleic acid construct encoding a protein to cells or tissues. Therefore, the invention includes a method of administering a protein or a nucleic acid encoding an protein that is an activator of a chitinase-like protein molecule. (Sambrook et al., 1989, Molecular Cloning: A Laboratory Manual, Cold Spring Harbor Laboratory, New York; Ausubel et al., 1997, Current Protocols in Molecular Biology, John Wiley & Sons, New York).

One of skill in the art will realize that diminishing the amount or activity of a molecule that itself diminishes the amount or activity of a chitinase-like protein molecule can serve to increase the amount or activity of a chitinase-like protein molecule. Antisense oligonucleotides are DNA or RNA molecules that are complementary to some portion of an mRNA molecule. When present in a cell, antisense oligonucleotides hybridize to an existing mRNA molecule and inhibit translation into a gene product. Inhibiting the expression of a gene using an antisense oligonueleotide is well known in the art (Marcus-Sekura, 1988, Anal, Biochem. 172:289), as are methods of expressing an antisense oligonucleotide in a cell (Inoue, U.S. Pat. No. 5,190,931). The methods of the invention include the use of antisense oligonucleotide to diminish the amount of a molecule that causes a decrease in the amount or activity of a chitinase-like protein molecule, thereby increasing the amount or activity of a chitinase-like protein molecule. Contemplated in the present invention are antisense oligonucleotides that are synthesized and provided to the cell by way of methods well known to those of ordinary skill in the art. As an example, an antisense oligonucleotide can be synthesized to be between about 10 and about 100, more preferably between about 15 and about 50 nucleotides long. The synthesis of nucleic acid molecules is well known in the art, as is the synthesis of modified antisense oligonucleotides to improve biological activity in comparison to unmodified antisense oligonucleotides (Tullis, 1991, U.S. Pat. No. 5,023,243).

Similarly, the expression of a gene may be inhibited by the hybridization of an antisense molecule to a promoter or other regulatory element of a gene, thereby affecting the transcription of the gene. Methods for the identification of a promoter or other regulatory element that interacts with a gene of interest are well known in the art, and include such methods as the yeast two hybrid system (Bartel and Fields, eds., In: The Yeast Two Hybrid System, Oxford University Press, Cary, N.C.).

Alternatively, inhibition of a gene expressing a protein that diminishes the level or activity of a chitinase-like protein molecule can be accomplished through the use of a ribozyme. Using ribozymes for inhibiting gene expression is well known to those of skill in the art (see, e.g., Cech et al., 1992, J. Biol. Chem. 267:17479; Hampel et al., 1989, Biochemistry 28: 4929; Altman et al., U.S. Pat. No. 5,168,053). Ribozymes are catalytic RNA molecules with the ability to cleave other single-stranded RNA molecules. Ribozymes are known to be sequence specific, and can therefore be modified to recognize a specific nucleotide sequence (Cech, 1988, J. Amer. Med. Assn. 260:3030), allowing the selective cleavage of specific mRNA molecules. Given the nucleotide sequence of the molecule, one of ordinary skill in the art could synthesize an antisense oligonucleotide or ribozyme without undue experimentation, provided with the disclosure and references incorporated herein.

One of skill in the art will appreciate that activators of chitinase-like protein molecule gene expression can be administered singly or in any combination thereof. Further, chitinase-like protein molecule activators can be administered singly or in any combination thereof in a temporal sense, in that they may be administered simultaneously, before, and/or after each other. One of ordinary skill in the art will appreciate, based on the disclosure provided herein, that chitinase-like protein molecule activators to inhibit gene expression can be used to treat oxidant-mediated acute lung injury, and that art activator can be used alone or in any combination with another activator to effect a therapeutic result.

It will be appreciated by one of skill in the art, when armed with the present disclosure including the methods detailed herein, that the invention is not limited to treatment of acute lung injury once the acute lung injury is established. Particularly, the symptoms of the acute lung injury need not have manifested to the point of detriment to the subject; indeed, the acute lung injury need not be detected in a subject before treatment is administered. That is, significant pathology from an acute lung injury does not have to occur before the present invention may provide benefit. Therefore, the present invention, as described more fully herein, includes a method for preventing an acute lung injury in a subject, in that a chitinase-like protein molecule activator, as discussed previously elsewhere herein, can be administered to a subject prior to the onset of an acute lung injury, thereby preventing the acute lung injury as demonstrated by the data disclosed herein.

One of skill in the art, when armed with the disclosure herein, would appreciate that the prevention of acute lung injury encompasses administering to a subject a chitinase-like protein molecule activator as a preventative measure against acute lung injury. As more fully discussed elsewhere herein, methods of increasing the level or activity of a chitinase-like protein molecule encompass a wide plethora of techniques for increasing not only chitinase-like protein molecule activity, but also for increasing expression of a nucleic acid encoding a chitinase-like protein molecule. Additionally, as disclosed elsewhere herein, one skilled in the art would understand, once armed with the teaching provided herein, that the present invention encompasses a method of preventing a wide variety of diseases where increase expression and/or activity of a chitinase-like protein molecule mediates treats or prevents the disease. Methods for assessing whether a disease relates to decreased levels or activity of a chitinase-like protein molecule are known in the art. Further, the invention encompasses treatment or prevention of such diseases discovered in the future.

The invention encompasses administration of a chitinase-like protein molecule or an activator of an chitinase-like protein molecule to practice the methods of the invention; the skilled artisan would understand, based on the disclosure provided herein, how to formulate and administer the appropriate chitinase-like protein molecule or chitinase-like protein molecule activator to a subject. Indeed, the successful administration of chitinase-like protein molecule or activator has been reduced to practice as exemplified herein. However, the present invention is not limited to any particular method of administration or treatment regimen. This is especially true where it would be appreciated by one skilled in the art, equipped with the disclosure provided herein, including the reduction to practice using an art-recognized model of oxidant-mediated acute lung injury, that methods of administering a chitinase-like protein molecules, or activators thereof, can be readily determined by one of skill in the pharmacological arts.

As used herein, the term “pharmaceutically-acceptable carrier” means a chemical composition with which an appropriate chitinase-like protein molecule, or an activator thereof, may be combined and which, following the combination, can be used to administer the appropriate chitinase-like protein molecule, or an activator thereof, to a subject.

The pharmaceutical compositions useful for practicing the invention may be administered to deliver a dose of between about 0.1 ng/kg/day and 100 mg/kg/day.

in various embodiments, the pharmaceutical compositions useful in the methods of the invention may be administered, by way of example, systemically, parenterally, or topically, such as, in oral formulations, inhaled formulations, including solid or aerosol, and by topical or other similar formulations. In addition to the appropriate chitinase-like protein molecule, or an activator thereof, such pharmaceutical compositions may contain pharmaceutically acceptable carriers and other ingredients known to enhance and facilitate drug administration. Other possible formulations, such as nanoparticles, liposomes, resealed erythrocytes, and immunologically based systems may also be used to administer an appropriate chitinase-like protein molecule, or an activator thereof, according to the methods of the invention.

### Pharmaceutical Compositions

Compounds which are identified using any method described herein as potential useful compounds for treatment and/or prevention of acute lung injury can be formulated and administered to a subject for treatment of acute lung injury disclosed herein are now described.

The invention encompasses the preparation and use of pharmaceutical compositions comprising a compound useful for treatment of acute king injury disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Pharmaceutical compositions that are useful in the methods of the invention may be prepared, packaged, or sold in formulations suitable for oral, rectal, vaginal, parenteral, topical, pulmonary, intranasal, buccal, intravenous, ophthalmic, intrathecal and other known routes of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate, Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets, Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intravenous, intramuscular, intracisternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers, Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares. Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, contain 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically dosages of the compound of the invention which may be administered to an animal, preferably a human, range in amount from about 0.01 mg to 20 about 100 g per kilogram of body weight of the animal. While the precise dosage administered will vary depending upon any number of factors, including, but not limited to, the type of animal and type of disease state being treated, the age of the animal and the route of administration. Preferably, the dosage of the compound will vary from about 1 mg to about 100 mg per kilogram of body weight of the animal. More preferably, the dosage will vary from about 1. mu.g to about 1 g per kilogram of body weight of the animal. The compound can be administered to an animal as frequently as several times daily, or it can be administered less frequently, such as once a clay, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

### Biomarker

Chitinase-like protein molecules, such as YKL-40, are readily detectable in the serum of healthy subjects, as well as those suffering from acute lung injury. The present invention contemplates the measurement and comparison of the chitinase-like protein molecule levels in a subject as a measure of acute lung cell injury. In one embodiment the level of the chitinase-like protein molecule in a subject can be used as a measure of the likelihood that a subject will suffer from acute lung disease upon exposure to an oxidant, such as oxygen at a level greater than about 21%. In another embodiment, the level of the chitinase-like protein molecule in a subject can be used as a measure of the severity of the acute lung injury that a subject is suffering from. In a further embodiment, the level of the chitinase-like protein molecule in a subject can be used as a measure of the likelihood that a subject will respond to a treatment for acute lung. In still a further embodiment, the level of the chitinase-like protein molecule in a subject can be used as a measure of how well a subject is responding to a treatment for acute lung disease. In another embodiment, the level of the chitinase-like protein molecule in a subject can be used as a measure of how well a subject is likely to respond to the prophylactic administration of a treatment of acute lung injury.

In various embodiments, the level of the chitinase-like protein molecule in a subject is compared with at least one comparator. In various embodiments, the comparator can be another healthy subject, another subject suffering from acute lung injury, an historical measure of the level of the chitinase-like protein molecule in at least one healthy subject, an historical measure of the level of the chitinase-like protein molecule in at least one subject suffering from acute lung injury, the level of the chitinase-like protein molecule in the same subject early during disease progression, the level of the chitinase-like protein molecule in the same subject later during disease progression, the level of the chitinase-like protein molecule in the same subject before a treatment is administered, or the level of the chitinase-like protein molecule in the same subject after a treatment is administered.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### The Chitinase-Like Proteins Breast Regression Protein-39 and YKL-40 Regulate Hyperoxia-Induced Acute Lung Injury

To evaluate the role that BRP-39/YKL-40 plays role in the pathogenesis of HALT, the hyperoxia-induced responses were assessed in wild-type (WT) mice, mice with null mutations of BRP-39 (BRP-39−/−), mice that overexpress YKL-40 in a lung-specific fashion, and mice that lack BRP-39 and produce transgenic YKL-40 only in the respiratory epithelium. To assess the applicability of the murine findings to humans, the levels of YKL-40 in tracheal aspirates from premature babies receiving oxygen supplementation for respiratory failure were assessed. These studies demonstrate that hyperoxia is a potent inhibitor of BRP-39 expression and production, and that BRP-39 and YKL-40 inhibit the toxic effects of hyperoxia. It was also demonstrated that the levels of tracheal YKL-40 are lower in premature babies that develop bronchopulmonary dysplasia (BPD) or die compared with those without these complications.

The studies described herein were designed to determine if the chitinase-like protein molecules, BRP-39 and YKL-40, regulate the ALI induced by the prototypic oxidant, 100% O₂. These studies demonstrate that hyperoxia inhibits BRP-39 expression and production in the otherwise naive murine lung and in epithelial cells in culture. They also demonstrate that mice that lack BRP-39 have exaggerated responses to 100% O₂, manifested by augmented alveolar-capillary permeability and protein leak, tissue oxidation, neutrophil- and macrophage-rich inflammation, chemokine elaboration, epithelial apoptosis, and premature death. Lastly, they demonstrate that transgenic YKL-40 ameliorates HALI, prolongs survival in 100% O₂, and rescues the exaggerated injury response in BRP-39^{-/-} animals. These studies highlight novel relationships between BRP-39/YKL-40 and oxidants in the lung, including the demonstration that oxidant injury decreases the expression and production of BRP-39, and that BRP-39 and YKL-40 are important inhibitors of oxidant-induced lung injury, permeability, and structural cell apoptosis.

BRP-39/YKL-40 is a product of the CH3L1 gene on chromosome 1 in mice and humans that is found in significant quantities in the circulation and tissues of normal humans and other animal species. BRP-39 and YKL-40 are also highly inducible, with elevated levels being seen in the serum and or tissues from patients with a variety of diseases, and elevated levels being noted in epithelial cells and/or macrophages after stimulation with IL-13 (Lee et al., 2009, J Exp Med 206:1149-1166) and during late stages of macrophage activation (Rehli et al., 2003, J Biol Chem 278:44058-44067). Transcriptional mechanisms have been shown to contribute to some of these stimulatory events (Rehli et al., 2003, J Biol Chem 278:44058-44067). Surprisingly, the present in vitro and in vivo studies are the first to highlight a circumstance in which the production and/or expression of BR-39/YKL-40 is decreased. They are also the first to define the relationship between this inhibition and oxidant injury, and to associate this decrease with a pathologic tissue response. The data disclosed here are consistent with the conclusion that a decrease in BRP-39/YKL-40 contributes to the initiation and/or perpetuation of the oxidant injury response.

Previous studies have shown that BRP-39 and YKL-40 inhibit the apoptosis of and CD95 expression by inflammatory cells at sites of Th2- and IL-13-induced inflammation (Lee et al., 2009, J Exp Med 206:1149-1166). It has also been demonstrated that acidic mammalian chitinase, a true chitinase, can also inhibit epithelial cell apoptosis, and that this inhibition is independent of the chitinolytic effects of the enzyme (Hart et al., 2009, J Immunol 182:5098-5106). The studies described herein have shown the relationship(s) between BRP-39/YKL-40 and apoptosis by demonstrating that these chilectins inhibit the oxidant-induced cell death of alveolar type H cells. Although not wishing to be bound by any particular theory, the findings are consistent with the phosphatidylinositol 3 kinase-Akt pathway being involved, because the present studies have demonstrated that BRP-39/YKL-40 is a potent activator of Akt (Lee et al., 2009, J Exp Med 206; 1149-1166), and Akt can confer cytoprotection in HALI (Waldow et al., 2008, Pulm Pharmacol Ther 21:418-429; Waxman et al., 2009, Am J Physiol Lung Cell Mol Physiol 41; 385-396; Xu et al., 2006, Am J Physiol Lung Cell Mol Physiol 291:L966-L975; Lu et al., 2001, J Exp Med 193:545-549). Thus, these studies suggest that BRP-39 and YKL-40 are critical regulators of cell death that inhibit oxidant injury and confer structural cell cytoprotection at physiologic concentrations, and prolong the survival of inflammatory cells, and contribute to antigen sensitization, chronic inflammation, and tissue remodeling when elevated.

ALI and ARDS are complex, multigenic, and multifactoral disorders, with profound clinical heterogeneity (Gao et al., 2009, Am J Physiol Lung Cell Mal Physiol 296:L713-L725). The studies described herein demonstrate that BRP-39 is inhibited during HALT and, in turn, feeds back to inhibit HALI. Recent studies have demonstrated that polymorphisms in the CH3L1 gene correlate with the levels of circulating YKL-40, the presence of asthma, and asthma severity (Chupp et al., 2007, N Engl J Med 357:2016-2027; Ober et al., 2008, N Engl J Med 358:1682-1691; Ober et al., 2009, Curr Opin Allergy Clin Immunol 9:401-408). The studies described herein suggest that polymorphisms in YKL-40 also play an important role in the pathogenesis of ALI and/or ARDS.

In animal models of ALI, inflammation and lung injury are frequently juxtaposed. This led to studies investigating the mechanisms of hyperoxia-induced inflammation, and the relationship between injury and inflammation in this disorder (Mantell et al., 1999, Ann N Y Acad Sci 887; 171-180; Bustani et al., 2003, Front Biosci 8:s694-s704; Lian et al., 2005, J Immunol 174:7250-7256). The studies described herein highlight an interesting relationship between the cell death and inflammatory responses in this model system.

The materials and methods employed in this Example are now described.

### Materials and Methods

Genetically Modified Mice

BRP-39^{-/-} mice were generated and used as previously described (Lee et al., 2009, J Exp Med 206; 1149-1166). The mice were generated on a mixed 129/C57BL/6 background and subsequently bred for more than 10 generations onto a C57BL/6 background. Transgenic mice in which human YKL-40 was tightly and inducibly overexpressed (CC10-rtTA-tTS-YKL-40) in a lung-specific manner were generated with constructs and approaches that have been previously described. (Lee et al., 2009, J Exp Med 206:1149-1166). Mice that lacked BRP-39 and produced YKL-40 only in pulmonary epithelial cells (CC10-rtTA-tTS-YKL-40/BRP-39^{-/-}) were generated by breeding the CC10-rtTA-tTS-YKL-40 and BRP-39^{-/-} mice. Mice with caspase-3-null mutations were kindly provided by Dr. Flavell (Dept. of Immunobiology, Yale University School of Medicine). Animal protocols were approved by the Yale University Institutional Animal Care and Use Committee, the guidelines of which were followed for all experiments.

Oxygen Exposure

Mice (4-6 wk old) were placed in cages in an airtight Plexiglas chamber (55×40×50 cm), as described previously (Waxman et al., 1998, J Clin Invest 101:1970-1982; Ward et al., 2000, Am J Respir Cell Mol Biol 22:535-542; Wang et al., 2003, J Biol Chem 278:31226-31232). Throughout the experiment, they were given free access to food and water. Oxygen levels were constantly monitored by an oxygen sensor, which was connected to a relay switch incorporated into the oxygen supply circuit. The inside of the chamber was kept at atmospheric pressure, and mice were exposed to a 12-hour light-dark cycle.

Bronchoalveolar Lavage

Mice were killed, the trachea was isolated by blunt dissection, and a small-caliber tube was inserted into the airway and secured. Two volumes of 1 ml of phosphate-buffered saline (PBS) containing 0.1% bovine serum albumin were instilled, gently aspirated, pooled, and processed as previously described (Come J et al., 2000, J Clin Invest 106:783-791; Waxman et al., 1998, J Clin Invest 101:1970-1982; Crapo et al., 1986, Anna Rev Physiol 48:721-731).

Immunohistochemistry

Immunohistochemistry (INC) was undertaken with a polyclonal anti-BRP-39, as previously described. (Homer et al., 2006, Am J Physiol Lung Cell Mol Physiol 291:L502-L511). Antibodies against surfactant apoprotein C (Millipore, Billerica, Mass.) and CC10 (Santa Cruz Biotechnology, Inc., Santa Cruz, Calif.) were used to identify alveolar type II cells and airway epithelial cells, respectively. The specificity of the staining was evaluated in experiments in which the primary antiserum was not used and experiments that compared tissue samples from WT and BRP-39^{-/-} animals.

Histological Analysis

The lungs were removed en bloc, inflated at 25-cm pressure with PBS containing 0.5% low melting point agarose, fixed in Streck solution (Streck, Omaha, Nebr.), embedded in paraffin, sectioned, and stained. Hematoxylin and eosin and Periodic acid-Schiff (PAS) stains were performed in the Research Histology Laboratory of the Department of Pathology at the Yale School of Medicine.

Immunoblot Analysis

Bronchoalveolar lavage (BAL; 50 μg) fluids and/or lung lysates were subjected to immunoblot analysis with antibodies against inhibitor of caspase-activated deoxyribonuclease (Chemicon International, Billerica, Mass.), caspase-3 (Cell Signaling Technology, Danvers, Mass.) or β-tubulin (Santa Cruz Biotechnology, Inc.), and the polyclonal rabbit antiserum against BRP-39, as noted above. These samples were fractionated by polyacrylamide gel electrophoresis, transferred to membranes, and evaluated as described previously (Kang et al., 2007, J Exp Med 204:1083-1093).

Quantification of BRP-39, CXCL-1, and CCL-2

The levels of BRP-39 in BAL or lung lysates were evaluated by ELISA with an anti-BRP-39 rabbit polyclonal IgG for capture and biotinylated anti-BRP-39, followed by horseradish peroxidase-labeled streptavidin (GE Healthcare, Piscataway, N.J.) for detection. This assay detects as little as 50 pg/ml recombinant BRP-39. The levels of BAL fluid CXCL-1 and CCL-2 were measured by ELISA with commercial kits (R&D Systems, Minneapolis, Minn.), as directed by the manufacturer.

mRNA Analysis

mRNA levels were assessed by real-time reverse transcriptase-polymerase chain reaction, as previously described. (Ma et al., 2006, J Clin Invest 116:1274-128; Lee et al., et al., 2004, J Exp Med 200:377-389). The sequences for the primers that were used were obtained from Primer Bank online (pga.mgh.harvard.edu/primerbank)

Terminal Deoxynucleotidyl Transferase-Mediated dUTP Nick End Labeling Staining

DNA fragmentation and cell death were evaluated with terminal deoxynucleotidyl transferase-mediated dUTP nick end labeling (TUNEL) assays, as previously described. (Lee et al., et al., 2004, J Exp Med 200:377-389).

8-Hydroxy-2′Deoxyguanosine Staining

Immunohistochemical detection of 8-hydroxy-2′deoxyguanosine (8-OHdG) was done using paraffin-embedded sections. The primary antibody used was mouse anti-8-OHdG (Santa Cruz Biotechnology, Inc., Santa Cruz, Calif.), The DAKO ARK system (Carpinteria, Calif.) was used as per the manufacturer's instructions. Negative controls consisted of isotype-matched control or rabbit serum.

Measurement of Tracheal Aspirate YKL-40

The tracheal aspirate samples were collected from neonates admitted to the Yale-New Haven Children's Hospital Newborn Special Care Unit. All infants had respiratory distress syndrome (RDS), which required them to be intubated, and administered at least one dose of natural surfactant and ventilated for treatment, per standard nursery guidelines. BPD was defined as the need for oxygen with characteristic radiographic changes at 36-weeks postmenstrual age. Samples were stored at −70° C. until assayed with ELISA. All human work was approved by the Human Investigational Committee at Yale University School of Medicine.

In Vitro Oxygen Exposure

Human bronchial epithelial cell line BEAS-2B cells (ATCC, Rockville, Md.) were incubated in complete bronchial epithelial basal medium (Lonza, Walkersville, Md.). They were placed in an airtight modular incubator chamber (Billups-Rothenberg, Del Mar, Calif.), which had been flushed continuously with 95% O₂/5% CO₂ until the oxygen level inside the chamber reached approximately 95%. The incubator chamber was then placed in a tissue culture incubator at 37° C., the O₂ inside the chamber was replaced every 24 hours, and the cells were harvested at the desired time points (24-72 h). The responses in these cells were compared with events in cells incubated in 5% CO₂ and air. In select experiments, the cells were incubated with N-acetyl-L-cysteine (Sigma, St. Louis, Mo.), which was added at a dose of 10 mM 1 hour before oxygen exposure, or its vehicle control.

Statistical Analysis

All data were initially checked for normal/parametric distribution (Kolmogorov-Smimov test). If parametric distribution was found, analysis of variance was applied to screen for differences among at least three groups. To compare two individual groups, Student's t test was applied. If nonparametric distribution was found, the Kruskal-Wallis test was applied to screen for differences among at least three groups, followed by the Mann-Whitney U test (Wilcoxon rank-sum test) to compare two individual groups. The survival rate was analyzed by the Kaplan-Meier method. Statistical significance was defined at a P value less than 0.05. All statistical analyses were performed with SPSS version 13.0 (SPSS Inc., Chicago, Ill.).

The results of this Example are now described.

### Hyperoxia Inhibition of BRP-39 In Vivo

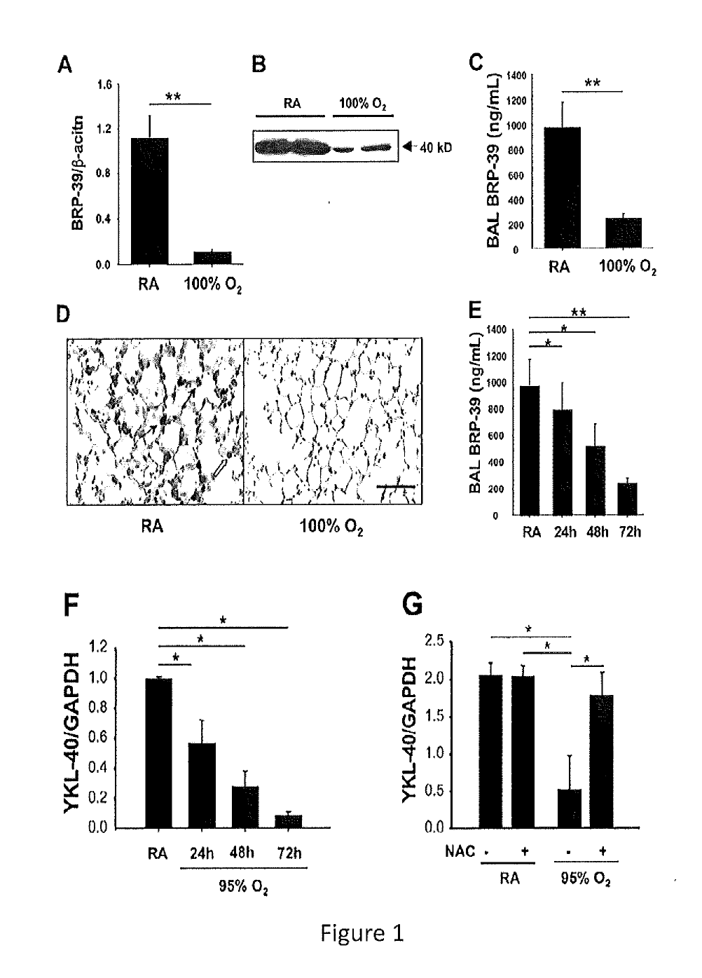

To begin to address the present hypothesis, the expression of BRP-39 in lungs from WT mice exposed to room air (RA) and 100% O₂. BRP-39 mRNA was readily appreciated in lungs were compared as depicted in FIG. 1A, and BRP-39 protein was abundant in BAL fluids from mice breathing RA as depicted in FIGS. 1B and 1C. IHC demonstrated that this BRP-39 was most readily appreciated in pulmonary macrophages and alveolar type 2 cells from these mice as depicted in FIG. 1D. In contrast, hyperoxia caused a significant decrease in BRP-39 mRNA and lung, macrophage, and alveolar type 2 cell BRP-39 protein accumulation as depicted in FIGS. 1A through 1D. This inhibition was seen after as little as 24 hours, and was most prominent after 72 hours of exposure to 100% O₂ as depicted in FIG. 1E. It was only partially BRP-39 specific, because similar inhibition of chitotriosidase expression was noted. These studies demonstrate that hyperoxia inhibits macrophage and epithelial cell BRP-39 expression and accumulation in the murine lung.

### Hyperoxia Inhibition of BRP-39 In Vitro

To further define the mechanism by which hyperoxia inhibits BRP-39/YKL-40, the expression of YKL-40 in BEAS-2B cells in RA or 95% oxygen were quantified, and the effects of antioxidants on these regulatory events were evaluated. In keeping with the present in vivo findings, hyperoxia inhibited YKL-40 expression in a time-dependent manner as depicted in FIG. 1F. Importantly, pretreatment with the antioxidant, N-acetyl-L-cysteine, abrogated the YKL-40-inhibiting effects of hyperoxia as depicted in FIG. 1G. These studies demonstrate that hyperoxia inhibits epithelial cell YKL-40 expression via an oxidant-dependent mechanism.

### BRP-39 Inhibition of HALI

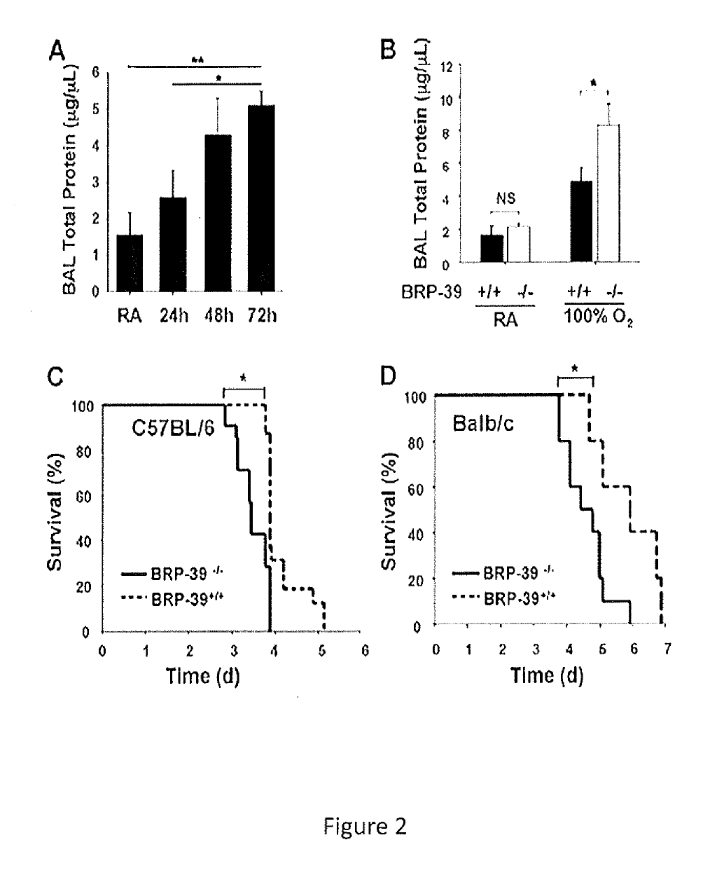

Next the effects of 100% O₂ in WT and BRP-39^{-/-} mice were compared. As previously described, (Waxman et al., 1998, J Clin Invest 101:1970-1982; He et al., 2005, J Clin Invest 115:1039-1048; Ward et al., 2000, Am J Respir Cell Mol Biol 22:535-542; Mantell et al., 1999, Ann N Y Acad Sci 887:171-180; Barazzone et al., 1998, Am J Respir Cell Mol Biol 19:573-581; O'Reilly et al., 2000, Lab Invest 80:1845-1854; Wang et al., 2003; J Biol Chem 278:29184-29191), exposure to 100% O₂ caused ALI, with alveolar capillary permeability alterations characterized by increased levels of BAL protein. This alveolar capillary protein leak was seen after as little as 24 hours, and progressed over the first 72 hours of 100% O₂ exposure (FIGS. 2A and 2B). With continued exposure, these lesions progressed, causing WT mice to begin to expire after 4 days of 100% O₂ exposure as depicted in FIGS. 2C and 2D. Interestingly, BRP-39^{-/-} mice had an exaggerated response to hyperoxia. This manifested as enhanced protein leak as depicted in FIG. 2B and premature death after 100% O₂ exposure as depicted in FIGS. 2C and 2D. These responses were not strain specific, because augmented BALI and accelerated death were seen in BRP-39^{-/-} mice on C57BL/6 and Balb/c backgrounds (FIGS. 2C and 2D and data not shown). Thus, BRP-39 is an important inhibitor of HALI.

### BRP-39 Inhibition of Hyperoxia-Induced Inflammation and Cytokine Production

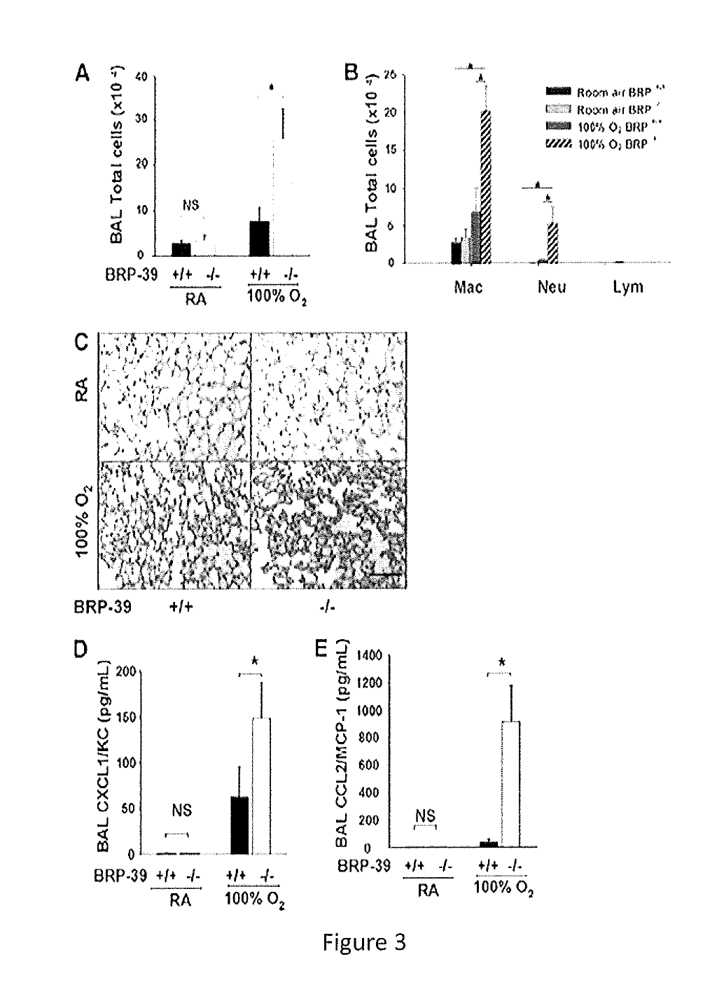

The BAL and tissue inflammatory responses in WT and BRP-39^{-/-} mice breathing RA or 100% O₂ were also compared. In WT mice, hyperoxia increased BAL total cell, macrophage, and neutrophil recovery, and induced a neutrophil- and macrophage-rich tissue inflammatory response as depicted in FIGS. 3A through 3C. In accord with their chemotactic properties for neutrophils and macrophages, these alterations were associated with significant increases in the levels of keratinocyte chemoattractant (KC)/CXCLI and monocyte chemoattractant protein (MCP)-1/CCL-2, respectively as depicted in FIGS. 3D and 3E. Importantly, BRP-39 played an important role in these alterations, because BAL total cell, macrophage and neutrophil recovery, tissue inflammation, and KC/CXCL1 and MCP-1/CCL-2 production were enhanced in BRP-39^{-/-} mice breathing 100% O₂ as depicted in FIGS. 3A-3E. These studies demonstrate that BRP-39 also inhibits hyperoxia-induced tissue inflammation and chemokine production.

### BRP-39 Inhibition of Hyperoxia-Induced Oxidant Injury and Apoptosis

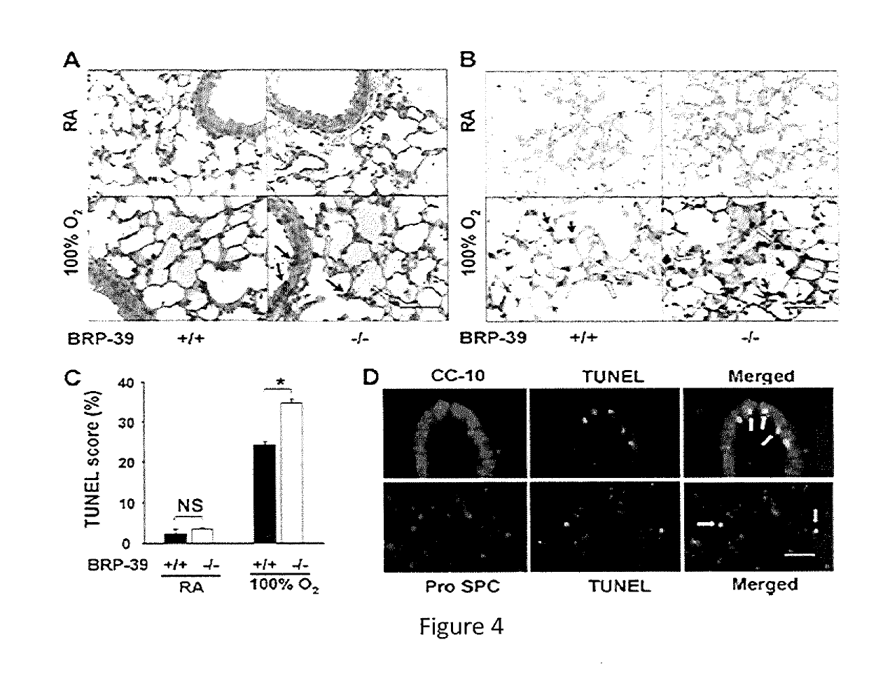

Because oxidant-induced DNA injury and cell death play important roles in the pathogenesis of HALI (Waxman et al., 1998, J Clin Invest 101:1970-1982; He et al., 2005, J Clin Invest 115:1039-1048), the roles of BRP-39 in these responses were evaluated. In accord with this concept, 100% O₂ caused oxidant injury that was readily apparent with 8-OHdG tissue staining as depicted in FIG. 4A, and DNA injury and cell death that manifested as increased levels of tissue TUNEL staining and caspase-3 activation as depicted in FIGS. 4B through 4D. BRP-39 played a critical role in the pathogenesis of both responses, because oxidant-induced tissue injury (8-OHdG staining), TUNEL staining, and caspase-3 activation were all significantly increased in BRP-39^{-/-} mice compared with WT mice as depicted in FIGS. 4A through 4D. Combined TUNEL and cell-specific IHC highlighted the alveolar and epithelial cell apoptosis in the BRP-39^{-/-} mice in 100% O₂ as depicted in FIG. 4D. Thus, BRP-39 is a critical mediator of hyperoxia-induced oxidant injury, DNA injury, and cell death.

### Transgenic YKL-40 Inhibition of HALI

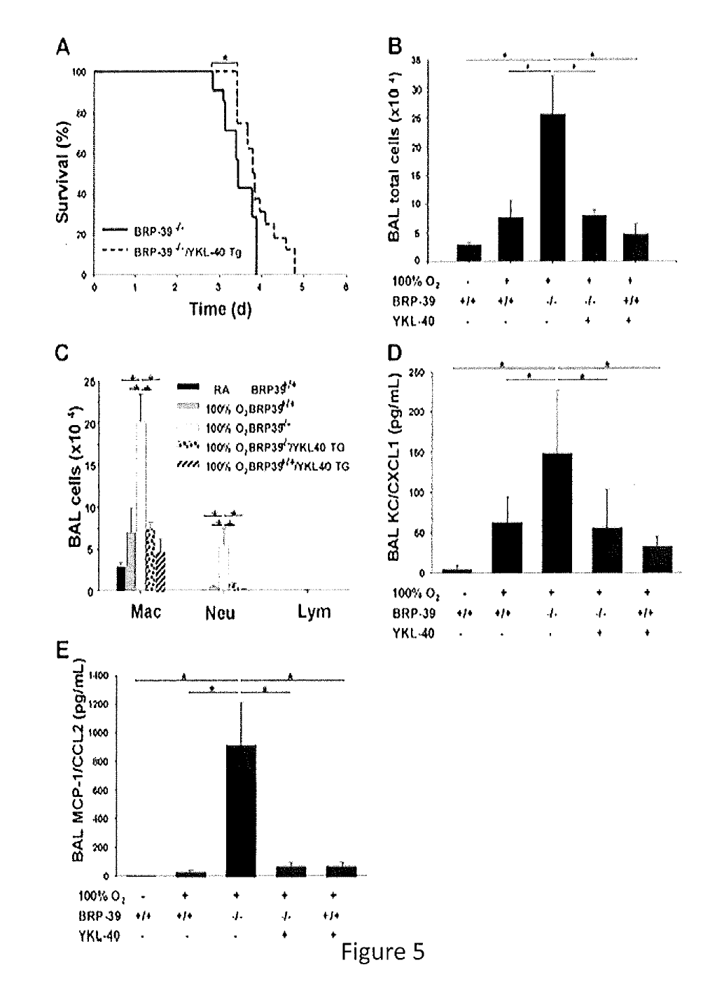

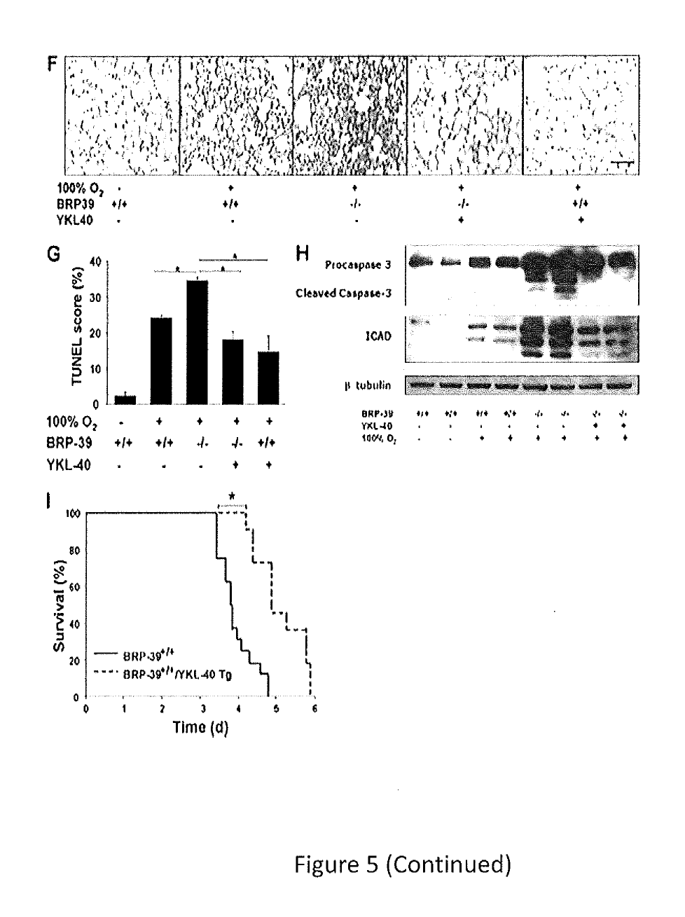

To further define the effector functions of BRP-39 and its human homolog, YKL-40, two approaches were used. In the first, the CC10-rtTA-tTS-YKL-40 mice were bred with the BRP-39^{-/-} mice to generate CC10-rtTA-tTS-YKL-40/BRP-39^{-/-} (BRP-39^{-/-}/YKL-40 Tg) mice that did not produce BRP-39, and only produced YKL-40 in the epithelium of the lung. This allowed the evaluation of the effects of YKL-40 in hyperoxia in the absence of potentially confounding responses induced by endogenously produced BRP-39, Transgenic YKL-40 enhanced the survival of BRP-39^{-/-} mice exposed to 100% oxygen as depicted in FIG. 5A. It also demonstrated the ability of YKL-40 to rescue the augmented hyperoxia-induced responses in BRP-39^{-/-} animals. As noted previously here, 100% O₂ caused exaggerated HALT responses, with alveolar capillary permeability alterations, protein leak, tissue and BAL neutrophil- and macrophage-rich inflammation, KC/CXCL-1 and MCF-1/CCL2 production, a TUNEL-positive cell death response, and caspase-3 activation in BRP-39^{-/-} mice as depicted in FIGS. 5B through 5H. These augmented responses in BRP 39^{-/-} mice were restored to levels comparable to those in WT animals by epithelial-targeted transgenic YKL-40 as depicted in FIGS. 5B-5H.

Experiments were also undertaken with CC10-rtTA-tTS-YKL-40 mice on a WT genetic background. These experiments compared the hyperoxia-induced responses in mice with physiologic and supraphysiologic levels of BRP-39/YKL-40. The features of the HALL that were seen in WT mice with physiologic levels of BRP-39 have been described previously. Importantly, survival was enhanced as depicted in FIG. 5I and the alveolar capillary permeability alterations, protein leak, tissue and BAL inflammation, KC/CXCL-1 and MCP-1/CCL2 production, TUNEL-positive cell death, and caspase-3 activation were diminished in the YKL-40 Tg animals in 100% O₂ as depicted in FIGS. 5B through 5H. When viewed in combination, these studies demonstrate that epithelial-targeted YKL-40 inhibits the toxic manifestations of 100% O₂ in the murine lung, and abrogates the exaggerated HALI in lungs from BRP-39^{-/-} mice.

### Caspase-3 Drives Inflammation in BRP-39^{-/-} Mice Exposed to 100% O₂

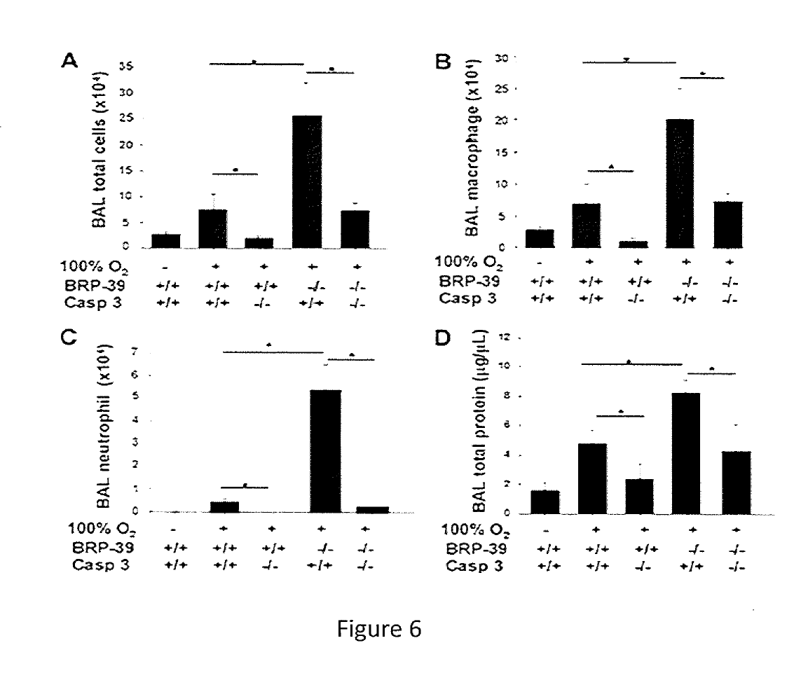

To understand the relationship between the inflammation and permeability and caspase activation in the present modeling system, BRP-39^{-/-} and caspase-3^{-/-} mice were bred and the effects of 100% O₂ in WT mice, single mutant mice, and mice with null mutations of BRP-39 and caspase-3 (BRP-39^{-/-} caspase-3^{-/-}) were compared. As noted previously here, hyperoxia caused a neutrophil- and macrophage-rich inflammatory response in WT mice, which was exaggerated in BRP-39^{-/-} mice. Interestingly, BAL and tissue inflammation were markedly ameliorated in the BRP-39^{-/-}/caspase-3^{-/-} mice as depicted in FIGS. 6A-6C. Similarly, hyperoxia caused alveolar capillary protein leak in WT mice, which was exaggerated in BRP-39^{-/-} animals as depicted in FIG. 6D. Interestingly, the hyperoxia-induced permeability changes in WT mice, and the exaggerated permeability alterations in BRP-39^{-/-} mice, were markedly ameliorated in mice that lacked caspase-3 as depicted in FIG. 6D. These studies demonstrate that caspase-3, a critical effector of apoptosis, plays an essential role in the pathogenesis of the exaggerated inflammation and permeability alterations in hyperoxia-exposed WT and BRP-39^{-/-} animals.

### Differences in the Levels of Tracheal Aspirate YKL-40 in Premature Newborns on Supplemental Oxygen

RDS and respiratory failure are problematic consequences of premature birth. Patients with these conditions are commonly treated with mechanical ventilation, supplemental oxygen, and surfactant preparations (Ramanathan et al., 2008, Neonatology 93:302-308), and, in many cases, rapidly recover. However, in a subset of patients, oxidant injury contributes to the development of BPD with chronic respiratory failure, and death can ensue (Bhandari et al., 2009, Pediatrics 123:1562-1573). To determine if the present murine findings are relevant to human disease, the levels of tracheal aspirate YKL-40 in a cohort of premature babies with RDS that developed BPD or died, and premature infants with milder disease that did not experience these adverse consequences were compared. In this cohort, the premature infants with the milder disease had higher levels of this chitinase-like protein molecule. These observations are in accord with the present findings that YKL-40 inhibits HALI. They also raise the possibility that the elevated levels of YKL-40 are causally related to the milder disease in these individuals. Oxidant injury also plays a major role in the pathogenesis of interstitial lung diseases, asthma, and chronic obstructive pulmonary disease and can worsen the effects of pulmonary infections (Tateda et al., 2003, J Immunol 170:4209-4216; Tuder et al., 2003, Am J Respir Cell Mol Biol 29:88-97; Andreadis et al., 2003, Free Radio Biol Med 35:213-225; Rahman et al., 2003, J Biochem Mol Biol 36:95-109; Saleh et al., 1997, Am J Respir Crit Care Med 155:1763-1769).

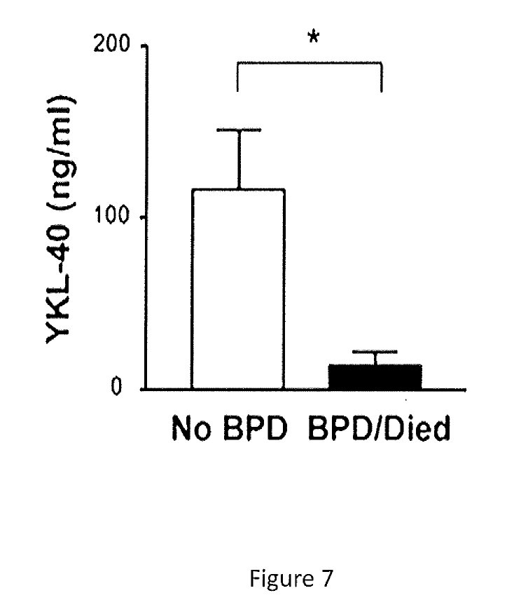

To determine if the present murine findings are relevant to humans, the levels of YKL-40 in tracheal aspirates from premature newborns on mechanical ventilation and supplemental oxygen due to respiratory failure were measured. Because oxidant injury is known to contribute to the pathogenesis of BPD (Davis et al., 2002, Acta Paediatr Suppl 91:23-25), the tracheal YKL-40 in infants that developed BPD or died were compared with those that did not develop these complications. Although there was no statistically significant difference between the “no BPD” (n=4) and BPD/death (n=5; two deaths) groups in the use of antenatal steroids, delivery route, percent male sex, gestational age, and the degree of oxygen supplementation (maximum Fi_{O2}), their birth weights were significantly different as shown in Table 1. There were also no significant differences in the two groups in terms of Apgar scores at 1 and 5 minutes and the time of collection of tracheal aspirate samples. Expectedly, babies in the BPD/death group had higher indices of severity of lung disease, as exemplified by more doses of surfactant and longer duration of invasive mechanical ventilation and exposure to supplemental oxygen as shown in Table 1. YKL-40 was readily apparent in aspirates from premature babies being ventilated for RDS. Interestingly, the levels of tracheal aspirate YKL-40 were significantly lower in the babies that subsequently developed BPD or death compared with the babies that did not develop these outcomes (P=0.01) as depicted in FIG. 7. These studies demonstrate that elevated levels of tracheal aspirate YKL-40 are associated with improved pulmonary outcomes in premature neonates that are on supplemental oxygen due to respiratory failure.

When viewed in combination, these observations show that BRP-39/YKL-40 can be manipulated to control oxidant-induced pulmonary responses, and that the levels of circulating and or organ YKL-40 are useful biomarkers that can predict the severity and or course of these disorders. For example, in premature newborns, YKL-40 can be used as a therapeutic in infants with RDS to prevent or ameliorate BPD, and the levels of tracheal aspirate YKL-40 might predict who will develop BPD and who will not. These studies also suggest that genetic polymorphisms, environmental exposures, or pharmacologic interventions that alter the levels and/or effects of BRP-39/YKL-40 can have major effects on an individual's ability to tolerate an oxidative load, and thus contribute to the severity and/or natural history of these disorders.

TABLE 1
Demographic and clinical characteristics of the study groups (no
bronchopulmonary dysplasia and bronchopulmonary dysplasia or died)

| | No BPD | BPD or Died | |
|---|---|---|---|
| | (n = 4) | (n = 5) | P Value |
| Prenatal steroids, % | 3 (75) | 5 (100) | 0.44 |
| C-section delivery, % | 3 (75) | 5 (100) | 0.44 |
| Male sex, % | 1 (25) | 3 (60) | 0.52 |
| Gestational age, wk | 26.2 ± 1.8 | 26.3 ± 1.6 | 0.92 |
| Birth weight, g | 898 ± 111 | 704 ± 81 | 0.02 |
| Apgar @ 1 min ≦3, % | 1 (25) | 2 (40) | 1.00 |
| Apgar @ 5 min ≦7, % | 1 (25) | 1 (20) | 1.00 |
| TA sample collected, d | 2.3 ± 1.3 | 2.4 ± 1.7 | 0.89 |
| Maximum Flₒ_{₂} (on TA | 0.27 ± 0.02 | 0.30 ± 0.04 | 0.49 |
| sample collection day) | | | |
| Survanta, doses | 1.0 ± 0.0 | 2.2 ± 0.8 | <0.05 |
| ETT PPV, d | 7.3 ± 8.8 | 43.8 ± 11.4 | 0.001 |
| Oxygen, d | 28.8 ± 10.1 | 68.0 ± 16.9 | 0.005 |
| Length of hospitalization, d | 65.0 ± 14.5 | 68.4 ± 16.9 | 0.76 |

Definition of abbreviations:
BPD = bronchopulmonary dysplasia;
ETT PPV = endotracheal tube positive pressure ventilation;
TA = tracheal aspirate.
Values are expressed as means (±SD).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of treating acute lung injury in a subject, said method comprising administering an effective amount of a chitinase-like protein molecule, or activator thereof, to said subject, thereby treating said acute lung injury.

2. The method of claim 1, wherein said chitinase-like protein molecule is YKL-40.

3. The method of claim 1, wherein said acute lung injury is oxidant-mediated acute lung injury.

4. The method of claim 1, wherein said subject is a human.

5. A method of preventing acute lung injury in a subject, said method comprising administering an effective amount of a chitinase-like protein molecule, or activator thereof, to said subject, thereby preventing said acute lung injury.

6. The method of claim 5, wherein said chitinase-like protein molecule is YKL0-40.

7. The method of claim 5, wherein said acute lung injury is oxidant-mediated acute lung injury.

8. The method of claim 5, wherein said subject is a human.

9. A method of determining the severity of acute lung injury in a subject, the method comprising:
a. obtaining a sample from the subject, wherein the subject has, or is suspected of having, acute lung injury,
b. determining in the sample the level of at least one chitinase-like protein molecule,
c. comparing the level of the at least one chitinase-like protein molecule in the sample with the level in a control or reference standard, wherein the difference in the level of the at least one chitinase-like protein molecule between the sample and the control or reference standard is a measure of the severity of acute lung injury in the subject.

10. The method of claim 9, wherein said chitinase-like protein molecule is YKL0-40.

11. The method of claim 9, wherein said acute lung injury is oxidant-mediated acute lung injury.

12. The method of claim 9, wherein said subject is a human.

13. A method of evaluating the progression of acute lung injury in a subject, the method comprising:
a. obtaining a sample from the subject, wherein the subject has, or is suspected of having, acute lung injury,
b. determining in the sample the level of at least one chitinase-like protein molecule,
c. comparing the level of the at least one chitinase-like protein molecule in the sample with the level in a control or reference standard at a first time point,
d. comparing the level of the at least one chitinase-like protein molecule in the sample with the level in a control or reference standard at a second time point, wherein the difference in the level of the at least one chitinase-like protein molecule between the sample and the control or reference standard at the first time point and the second time point is a measure of the progression of acute lung injury in the subject.

14. The method of claim 13, wherein said chitinase-like protein molecule is YKL-40.

15. The method of claim 13, wherein said acute lung injury is oxidant-mediated acute lung injury.

16. The method of claim 13, wherein said subject is a human.

17. A method of evaluating the effect of a treatment of acute lung injury in a subject, the method comprising:
a. obtaining a sample from the subject, wherein the subject has, or is suspected of having, acute lung injury,
b. determining in the sample the level of at least one chitinase-like protein molecule,
c. comparing the level of the at least one chitinase-like protein molecule in the sample with the level in a control or reference standard before treatment,
d. comparing the level of the at least one chitinase-like protein molecule in the sample with the level in a control or reference standard after treatment, wherein the difference in the level of the at least one chitinase-like protein molecule between the sample and the control or reference standard before treatment and after treatment is a measure of the effect of the treatment of acute lung injury on the subject.

18. The method of claim 17, wherein said chitinase-like protein molecule is YKL-40.

19. The method of claim 17, wherein said acute lung injury is oxidant-mediated acute lung injury.

20. The method of claim 17, wherein said subject is a human.
